(19) United States
(12) Patent Application Publication
(10) Pub. No.: US 2012/0230871 A1
(43) Pub. Date: Sep. 13, 2012

(54) Microbiocidal Control in Drinking Line Systems

(75) Inventor: Eric W. Liimatta, Baton Rouge, LA (US)

(73) Assignee: ALBEMARLE CORPORATION, Baton Rouge, LA (US)

(21) Appl. No.: 13/512,743

(22) PCT Filed: Nov. 22, 2010
(86) PCT No.: PCT/US2010/057586

§ 371 Date: May 30, 2012

Related U.S. Application Data
(60) Provisional application No. 61/266,687, filed on Dec. 4, 2009.

(51) Int. Cl. A61L 2/18

(52) U.S. Cl. 422/37

(57) ABSTRACT

This invention provides a method of controlling microbes in a drinking line system when poultry and/or swine are absent from the area where the drinking line system is located. The method comprises I) contacting the drinking line system and an aqueous microbiocidal solution; and II) flushing said drinking line system with water and/or an aqueous solution comprising one or more scale removers at least once after said contacting with said aqueous microbiocidal solution, wherein said aqueous microbiocidal solution is formed from components comprising water and a concentrated aqueous biocidal solution selected from A) an aqueous biocide solution having an active bromine content of about 50,000 ppm or more, which solution is formed from components comprising water and (i) bromine chloride or bromine chloride and bromine, with or without conjoint use of chlorine, and (ii) overbased alkali metal salt of sulfamic acid and/or sulfamic acid, alkali metal base, and water, wherein the relative proportions of (i) and (ii) are such that the atom ratio of nitrogen to active bromine is greater than 0.93, and wherein the pH of the composition is greater than 7, or B) an aqueous biocide solution formed from components comprising water and (i) at least one bromide source selected from ammonium bromide, hydrogen bromide, at least one alkali metal bromide, at least one alkaline earth metal bromide, and mixtures of any two or more of the foregoing, (ii) a chlorine source, optionally (iii) at least one inorganic base, and optionally (iv) sulfamic acid and/or a metal salt of sulfamic acid, or C) a combination of A) and B). Any degradation of the materials of the drinking line system from contact with said aqueous microbiocidal solution is minimal.

### TECHNICAL FIELD

This invention relates to microbiocidal control in drinking line systems with a halogen-based biocide.

### BACKGROUND

Drinking line systems for animals, particularly poultry and swine, need to be “clean”, that is, they should be free of microbial contamination, or contain only a minimal amount of microbial contamination. Microbes present in the drinking line systems are ingested by the poultry or swine when water is consumed from the drinking line, and can make the poultry bird or swine animal sick, often necessitating treatment of the poultry or swine with antibiotics. In addition, the contamination in drinking lines is often in the form of biofilm, which contain slime layers that protect the microbes, and thus biofilm is usually more difficult to control and eliminate.

Methods for controlling bacterial contamination in drinking line systems are known. Normally, the drinking line systems are periodically flushed with a microbiocidal substance. While concentrated bleach solutions are known to be effective at controlling the microbiocidal contamination in drinking line systems, bleach degrades the substances from which drinking nipples and water regulators are usually made. A peroxide-based treatment method that causes only minimal degradation of the drinking line system materials is known, but requires pre-mixing of two components immediately prior to administration, and special equipment to administer the treatment. It would be highly advantageous if a way could be found for treatment of drinking line systems that is effective at controlling microbes while avoiding or minimizing the degradation of the drinking line system materials; especially if such treatment is easy and economical to administer or carry out.

### SUMMARY OF THE INVENTION

This invention provides methods for cleaning drinking line systems, especially those for poultry and swine. In the practice of this invention, drinking line systems are cleaned via contact with an aqueous microbiocidal solution. Surprisingly, only minimal adverse effects on the materials of the drinking line systems have been observed, despite the fact that the biocides are bromine-based. In other words, any degradation of the materials of the drinking line system is such that the parts of the drinking line system can continue to perform their function properly after treatment with a bromine-based biocide in the practice of this invention. In addition, the bromine-based biocides integrate well with existing systems. Another advantage provided by the use of bromine-based biocides in the present invention is the avoidance of low pH values in the drinking line system; pH values in the range of 2 to 5 have been found to favor mold growth.

One embodiment of this invention is a method for controlling microbes in a drinking line system when poultry and/or swine are absent from the area where the drinking line system is located. The method comprises
I) contacting the drinking line system and an aqueous microbiocidal solution; and
II) flushing said drinking line system with water and/or an aqueous solution comprising one or more scale removers at least once after said contacting with said aqueous microbiocidal solution,
wherein said aqueous microbiocidal solution is formed from components comprising water and a concentrated aqueous biocidal solution selected from
A) an aqueous biocide solution having an active bromine content of about 50,000 ppm or more, which solution is formed from components comprising water and (i) bromine chloride or bromine chloride and bromine, with or without conjoint use of chlorine, and (ii) overbased alkali metal salt of sulfamic acid and/or sulfamic acid, alkali metal base, and water, wherein the relative proportions of (i) and (ii) are such that the atom ratio of nitrogen to active bromine is greater than 0.93, and wherein the pH of the composition is greater than 7, or
B) an aqueous biocide solution formed from components comprising water and (i) at least one bromide source selected from ammonium bromide, hydrogen bromide, at least one alkali metal bromide, at least one alkaline earth metal bromide, and mixtures of any two or more of the foregoing, (ii) a chlorine source, optionally (iii) at least one inorganic base, and optionally (iv) sulfamic acid and/or a metal salt of sulfamic acid, or
C) a combination of A) and B).
   Any degradation of the materials of the drinking line system from contact with said aqueous microbiocidal solution is minimal.

These and other embodiments and features of this invention will be still further apparent from the ensuing description and appended claims.

### FURTHER DETAILED DESCRIPTION OF THE INVENTION

As used throughout this document, the phrase “cleaning drinking line systems” refers to the treatment of drinking line systems to minimize or eliminate microbial contamination. Throughout this document, the term “drinking line system” refers to a system that includes at least diaphragms, drinking lines, and nipple assemblies, and may optionally include water regulators, water meters, medicators, slope compensators, shut-off valves, step regulators, stand tube/air breathers, and/or piping. A filter panel is usually included as part of the drinking line system, but the filter panel generally is not contacted by the aqueous microbiocidal solution. In the drinking line systems, medicators are typically made from polyvinyl chloride (PVC) or chlorinated polyvinyl chloride (CPVC); diaphragms are usually made from buna rubber, ethylene propylene diene monomer rubber (EPDM), or neoprene; piping is normally made from PVC; water regulators are typically made from CPVC; drinking lines are usually made from PVC; and nipple assemblies are normally made from plastic and a metal. The waterer of the nipple assembly for poultry is typically made from stainless steel (especially grades 302, 303, or 304) or brass; for swine, the waterer of the nipple assembly is normally made from stainless steel. Nipple assemblies are sometimes called drinking nipples.

As used throughout this document, the term “microbes” refers to bacteria, yeast, and mold, unless otherwise specified. Similarly, the phrase “microbial contamination”, as used throughout this document, refers to undesired growth of bacteria, yeast, and/or mold in drinking line systems.

Poultry birds are absent from the area where the drinking line system is located during the microbiocidal treatment of the drinking line system. Non-limiting examples of poultry that use drinking line systems include chicken, rooster, turkey, duck, goose, quail, pheasant, ostrich, game hen, emu, squab, guinea fowl, and Cornish hen. Swine animals are absent from the area where the drinking line system is located during the microbiocidal treatment of the drinking line system. Examples of swine that use drinking line systems include hogs, sows, gilts, barrows, boars, and pigs. The phrase “absent from the area where the drinking line system is located” means that the poultry and swine are not in the section or pen where the drinking line system is while that system or portion of the system undergoes treatment; in other words, the poultry and swine are prevented from accessing the drinking line system and consuming water from the drinking line system while the drinking line system is being treated. The water that the poultry and the swine drink is not treated pursuant to this invention.

In standard procedures for cleaning drinking line systems, a concentrated aqueous biocidal solution of a biocide is siphoned through a pump and mixed with water; the aqueous microbiocidal solution formed by the mixing of the water and the concentrated aqueous biocidal solution is used to treat the drinking line system. The pumping rate is set to provide a particular ratio of concentrated aqueous biocidal solution to water, usually about 1:128 (approximately one ounce per gallon). When a different concentration of the biocide in the aqueous microbiocidal solution, whether a higher or lower concentration, is desired, it is generally preferred to adjust the concentration of the biocide in the aqueous biocide solution, rather than to adjust the pumping rate, although adjustments to the pumping rate are feasible in the practice of this invention.

When the drinking line system is contacted with the aqueous microbiocidal solution, the aqueous microbiocidal solution is typically held in the drinking line system for a desired contact time, usually about one hour to about 36 hours, preferably about three hours to about 24 hours, after which the aqueous microbiocidal solution is usually flushed out of the drinking line system. A flushing with water and/or an aqueous solution comprising one or more scale removers (descalers), such as citric acid, is performed after the microbiocidal treatment to remove any residues of the aqueous microbiocidal solution from the system. When more than one flushing is performed, each flushing can be performed with water and/or an aqueous solution comprising one or more scale removers. For example, when two flushings are performed, one flushing can be with water and the other with an aqueous solution comprising one or more scale removers, or both flushings can be with water, or both with an aqueous solution comprising one or more scale removers. Optionally, the drinking line system can be flushed with water and/or an aqueous solution comprising one or more scale removers before treating the system with the aqueous microbiocidal solution. The flushing(s) can be performed at atmospheric pressure or at greater pressure. One advantage of performing at least one flushing at greater than atmospheric pressure is that it can help remove solid material that has deposited in the system.

The concentrated aqueous biocidal solutions of A) and B) above have bromine-based biocides therein, and these solutions thus have bromine residuals.

Aqueous biocide solutions of A) above are formed from water and (i) bromine chloride or bromine chloride and bromine, with or without conjoint use of chlorine, and (ii) overbased alkali metal salt of sulfamic acid (preferably a lithium, sodium, and/or potassium salt of sulfamic acid) and/or sulfamic acid, alkali metal base, and water. Preferably, the molar amount of chlorine is either equivalent to the molar amount of bromine or less than the molar amount of bromine, and a water-soluble source of sulfamate anion. The relative proportions of (i) and (ii) are such that the atom ratio of nitrogen to active bromine is greater than 0.93, and the pH of the concentrated aqueous biocidal solution is greater than 7.

When forming an aqueous biocide solution of A), the pH is normally at least 7 and preferably is always at a pH higher than 7, e.g., in the range of 10-14, by use of an inorganic base. Preferred bases are alkali metal bases, preferably an oxide or hydroxide of lithium, sodium, and/or potassium, more preferably sodium hydroxide and/or potassium hydroxide. If sulfamic acid is used in forming concentrated aqueous biocidal solution, the solution should also be provided with a base, preferably enough base to keep the solution alkaline, i.e., with a pH above 7, preferably above about 10 and most preferably about 13 or above.

It will be appreciated that even where the aqueous biocide solution of A) above is made from bromine chloride, a mixture of bromine chloride and bromine, or a combination of bromine and chlorine in which the molar amount of chlorine is either equivalent to the molar amount of bromine or less than the molar amount of bromine is used, the aqueous biocide solution is bromine-based as most of the chlorine usually forms chloride salts such as sodium chloride since an alkali metal base such as sodium hydroxide is typically used in the processing to raise the pH of the product solution to about 13 or greater. Thus the chlorine in the aqueous biocide solution of A) above is not present as a significant biocide.

The aqueous biocide solutions of A) have one or more active halogen species; preferred aqueous biocide solutions have one or more active bromine species. The active bromine content of the aqueous biocide solutions of A) is about 50,000 ppm (wt/wt) or more. Preferably, the aqueous biocide solution of A) has about 100,000 ppm (wt/wt) or more of active bromine, e.g., as much as about 105,000 to about 215,000 ppm of active bromine. Active halogen content is determinable by use of conventional starch-iodine titration. The pH of the aqueous biocide solution of A) above is greater than 7, preferably about 10 or greater, more desirably about 12 or greater, and still more desirably about 13 or greater. The atom ratio of nitrogen to active bromine in the aqueous biocide solution of A) above is greater than 0.93.

Processes for producing aqueous biocide solutions of A) are described in U.S. Pat. Nos. 6,068,861 and 6,299,909 B1. An aqueous biocide solution of A) containing over 50,000 ppm of active halogen is available commercially from Albemarle Corporation under the trademark SWG™ biocide (Albemarle Corporation); the pH of the aqueous product as received is normally in the range of 13 to 14.

Aqueous biocide solutions of B) above are formed from components comprising water and (i) at least one bromide source selected from ammonium bromide, hydrogen bromide, at least one alkali metal bromide, at least one alkaline earth metal bromide, and mixtures of any two or more of the foregoing, (ii) a chlorine source, optionally (iii) at least one inorganic base, and optionally (iv) sulfamic acid and/or a metal salt of sulfamic acid. Suitable bromide sources for forming aqueous biocide solutions of B) include ammonium bromide, hydrogen bromide, various suitable alkali metal bromides including LiBr, NaBr, KBr, and suitable alkaline earth metal bromides, viz., MgBr₂ and CaBr₂. Mixtures of two or more bromide sources can be used if desired. A preferred bromide source is NaBr. Suitable chlorine sources include hypochlorites, typically alkali metal hypochlorites or alkaline earth metal hypochlorites, solid chlorine sources, and chlorine (Cl₂). The aqueous biocide solutions of B) can include optionally (iii) at least one inorganic base, and optionally (iv) sulfamic acid and/or a metal salt of sulfamic acid.

There are several preferred combinations that form aqueous biocide solutions which fall within the definition of the aqueous biocide solutions of B) above.

Aqueous biocide solutions of a) are a preferred combination, and are formed from components comprised of water and (i) at least one bromide source as described above for B), (ii) a chlorine source which is at least one alkali metal hypochlorite and/or at least one alkaline earth metal hypochlorite, and (iii) an inorganic base. The interaction of these components results in an aqueous solution having a suitably high bromine residual. Suitable bromide sources for forming aqueous biocide solutions of a) are as described above for B). Mixtures of two or more bromide sources can be used if desired. A preferred bromide source is NaBr, especially NaBr from which trace amounts of alcohol such as methanol have been removed.

Various alkali metal hypochlorites or alkaline earth metal hypochlorites can be used to form the aqueous biocide solutions of a). Thus, use can be made of such materials as lithium hypochlorite, sodium hypochlorite, potassium hypochlorite, calcium hypochlorite, magnesium hypochlorite, and the like. Of such hypochlorites, use of sodium hypochlorite or calcium hypochlorite is most preferred. When using ammonium bromide as the bromide source in forming an aqueous biocide solution of a), it is desirable to employ therewith sodium hypochlorite in the manner described in U.S. Pat. No. 6,478,973. Several hypochlorite solutions are commercially available as articles of commerce since they are useful as bleaches, as well as intermediates for preparing other useful products. Metal bromides or hypochlorites of Be, Sr, or Ba should not be used because of toxicological concerns. Thus, the term “alkaline earth” as used herein excludes Be, Sr, and Ba.

If an excess amount of the hypochlorite is used relative to the amount of bromide salt used when forming the aqueous biocide solutions of a), the resultant solution will contain chlorine-based species as well as a bromine residual. These chlorine-based species are not harmful as long as the requisite quantity of bromine reserve is present in the solution being used. Preferably, any excess of hypochlorite is back-titrated with an aqueous alkali metal hypochlorite or alkaline earth metal hypochlorite so that the halogen reserve in the solution essentially consists of bromine reserve.

An inorganic base is used in the formation of the aqueous biocide solutions of a). Preferred bases are alkali metal bases, preferably an oxide or hydroxide of lithium, sodium, and/or potassium, more preferably sodium hydroxide and/or potassium hydroxide. When an inorganic base is used, the pH is normally about 7 or greater and preferably is higher than 7, e.g., a pH in the range of about 10 to about 14.

Sulfamic acid and/or a metal salt of sulfamic acid is optional but preferred in the aqueous biocide solutions of b). Metal salts of sulfamic acid are usually the alkali metal salts, including lithium sulfamate, sodium sulfamate, and potassium sulfamate. Sulfamic acid can be used alone or in a mixture with one or more metal salts of sulfamic acid. Sulfamic acid and/or sodium sulfamate are preferred.

A commercial aqueous biocide solution of a) that can be utilized in practicing this invention is available under the trade designation Stabrex® biocide (Nalco Chemical Company). This product contains active bromine stabilized against chemical decomposition and physical evaporation of active bromine species by the inclusion of sulfamate. For additional details concerning preparation of aqueous biocidal solutions of a) stabilized with sulfamic acid, see U.S. Pat. Nos. 6,007,726; 6,156,229; and 6,270,722.

Aqueous biocide solutions of b) are a preferred combination, and are formed from water and (i) at least one bromide source as described above for B), (ii) a chlorine source which is a solid chlorinating agent, and (iii) an inorganic base. Suitable bromide sources for forming aqueous biocide solutions of b) and the preferences therefor are as described above for B). Mixtures of two or more bromide sources can be used if desired. Suitable solid chlorinating agents include trichloroisocyanurate and sodium dichloroisocyanurate.

An inorganic base is used in the formation of the aqueous biocide solutions of b). Preferred bases are alkali metal bases, preferably an oxide or hydroxide of lithium, sodium, and/or potassium, more preferably sodium hydroxide and/or potassium hydroxide. When an inorganic base is used, the pH is normally about 7 or greater and preferably is higher than 7, e.g., a pH in the range of about 10 to about 14.

Sulfamic acid and/or a metal salt of sulfamic acid is optional but preferred in the aqueous biocide solutions of b). Metal salts of sulfamic acid are usually the alkali metal salts, including lithium sulfamate, sodium sulfamate, and potassium sulfamate. Sulfamic acid can be used alone or in a mixture with one or more metal salts of sulfamic acid. Sulfamic acid and/or sodium sulfamate are preferred.

An aqueous biocide solution of b) is available commercially under the trade designation BromMax® biocide (Enviro Tech Chemical Services, Inc.). This product contains active bromine stabilized against chemical decomposition and physical evaporation of active bromine species by the inclusion of sulfamate. For additional details concerning preparation of aqueous biocidal solutions of b) stabilized with sulfamic acid, see U.S. Pat. Nos. 7,045,153; 7,309,503; and 7, 455,859.

The aqueous biocide solutions of c) are a preferred combination, and are formed from water and (i) at least one bromide source as described above for B), (ii) a chlorine source, and (iv) sulfamic acid and/or a metal salt of sulfamic acid. Suitable bromide sources for forming aqueous biocide solutions of c) and the preferences therefor are as described above for B). Mixtures of two or more bromide sources can be used if desired.

The chlorine source to form the aqueous biocide solutions of c) can be chlorine and/or any of various alkali metal hypochlorites or alkaline earth metal hypochlorites. The hypochlorites can be any of those described above for a). Of such hypochlorites, sodium hypochlorite is most preferred.

Metal salts of sulfamic acid are usually the alkali metal salts, including lithium sulfamate, sodium sulfamate, and potassium sulfamate. Sulfamic acid can be used alone or in a mixture with one or more metal salts of sulfamic acid. Sulfamic acid is preferred.

An inorganic base is optional but preferred in the aqueous biocide solutions of c). Preferred bases are alkali metal bases, preferably an oxide or hydroxide of lithium, sodium, and/or potassium, more preferably sodium hydroxide and/or potassium hydroxide. When an inorganic base is used, the pH is normally about 7 or greater and preferably is higher than 7, e.g., a pH in the range of about 10 to about 14.

A commercial aqueous biocide solution of c) that can be utilized in practicing this invention is available under the trade designation Justeq07 biocide (Justeq, LLC). This product contains active halogen species stabilized by the inclusion of sulfamate. Processes for producing aqueous biocide solutions of c) are described in U.S. Pat. Nos. 6,478,972; 6,533,958; and 7,341,671.

The aqueous microbiocidal solutions, which contain bromine-based biocides, tend to be less odorous than chlorine-based microbiocides. Moreover, while some of the bromine-based microbiocides may possibly react with nitrogenous species which may be present, the resultant bromamines would also possess microbiological activity. Thus such side reactions would not materially decrease the microbiological effectiveness by use of these bromine-based microbiocides. Furthermore, bromamines generally do not exhibit obnoxious properties toward workers in the area, whereas chloramines resulting from use of certain chlorine-based microbiocides under the same conditions tend to be powerful lachrymators.

When combined with water to treat form the aqueous microbiocidal solution, the proportions of water and the concentrated aqueous biocidal solution are such that the concentration of the active halogen species provides a bromine residual in the range of about 50 to about 3200 ppm (wt/wt) as total bromine, preferably in the range of about 100 to about 2000 ppm (wt/wt) as total bromine, more preferably in the range of about 300 to about 1800 ppm (wt/wt) as total bromine, and still more preferably in the range of about 400 to about 1600 ppm (wt/wt) as total bromine. To achieve some of the higher bromine residuals in these ranges, it may necessary to adjust the pumping rate so that the ratio of concentrated aqueous biocidal solution to water is higher than 1:128.

The aqueous microbiocidal solution is typically used at a temperature of about 5 to about 39° C., but can be used at higher temperatures, e.g., up to about 43° C., if desired.

Other additives can be used in conjunction with the aqueous microbiocidal solution, provided that the other additive or additives are compatible with the aqueous microbiocidal composition, minimally degrade or do not degrade the materials of the drinking line system, and do not otherwise detract from the microbiocidal effectiveness of the aqueous microbiocidal solution in any appreciable manner. In general, additives which are compatible with aqueous hypochlorite bleach solutions such as certain radical scavengers, chelating agents, pH buffering agents, surfactants, and polymers as described in detail in U.S. Pat. No. 6,506,718 may be used, if desired. It is also possible to use one or more wetting agents, hydrotropes, thickeners, defoaming agents, foaming agents, dyes, and similar functional additives that meet the above criteria. If used, the amount of each suitable selected additive to be used in conjunction with the microbiocides used pursuant to this invention should be sufficient to provide the property for which it is employed. Recommendations from manufacturers of such additives are useful guidelines in this respect. When such other additive is included, it is usually present in the aqueous biocide solution prior to the siphoning of the aqueous biocide solution to be mixed with water to form the aqueous microbiocidal solution. Alternatively, such additives can be added to the water to be mixed with the aqueous biocide solution. The inclusion of certain ingredients, such as dyes and/or foaming agents, which can indicate that the drinking line system is filled with the aqueous microbiocidal solution, is preferred.

Suitable methods for determining “bromine residual” are known and reported in the literature. See for example, Standard Methods For the Examination of Water and Wastewater, 18th Edition, 1992, from American Public Health Association, 1015 Fifteenth Street, NW, Washington, D.C. 20005 (ISBN 0-87553-207-1), pages 4-36 and 4-37; Hach Water Analysis Handbook, Third Edition, 1997, by Hach Company, Loveland Colo., especially pages 1206 and 1207; and Handbook of Industrial Water Conditioning, 7th edition, Betz Laboratories, Inc., Trevose, Pa. 19047 (Library of Congress Catalog Card Number: 76-27257), 1976, pages 24-29.

The term “bromine residual” refers to the amount of bromine species present in the treated water available for disinfection. Residuals can be determined as either “total” or “free” depending upon the analytical test method employed. In the present case, the numerical values for bromine residual have been given herein mostly on a total bromine basis. Such values can be monitored by use of the analytical procedure for “total chlorine” given below. However if desired, the bromine residual could be monitored on a “free bromine” basis by using the analytical procedure for “free chlorine” given below. In either case the numerical values obtained are in terms of chlorine and thus such values are multiplied by 2.25 to obtain the corresponding bromine values. Typically the values on a “total bromine” basis on a given sample will be higher than the values on a “free bromine” basis on the same given sample. The important point to understand is that this invention relates to the bromine residual that is actually present in the treated aqueous medium whether the value is determined by use of the total chlorine test procedure or the free chlorine test procedure, but use of the total chlorine test procedure is recommended.

In order to measure the quantity of active bromine in water used in forming an aqueous microbiocidal composition of this invention, standard well known analytical procedures can be used. The term “active bromine” of course refers to all bromine-containing species that are capable of biocidal activity. It is generally accepted in the art that all of the bromine in the +1 oxidation state is biocidally active and is thus included in the term “active bromine”. As is well known in the art, bromine, bromine chloride, hypobromous acid, hypobromite ion, hydrogen tribromide, tribromide ion, and organo-N-brominated compounds have bromine in the +1 oxidation state. Thus these, as well as other such species to the extent they are present, constitute the active bromine content of the compositions of this invention. See, for example, U.S. Pat. No. 4,382,799 and U.S. Pat. No. 5,679,239. A well-established method in the art for determining the amount of active bromine in a solution is starch-iodine titration, which determines all of the active bromine in a sample, regardless of what species may constitute the active bromine. The usefulness and accuracy of the classical starch-iodine method for quantitative determination of bromine and many other oxidizing agents has long been known, as witness Chapter XIV of Willard-Furman, Elementary Quantitative Analysis, Third Edition, D. Van Nostrand Company, Inc., New York, Copyright 1933, 1935, 1940.

A typical starch-iodine titration to determine active bromine is carried out as follows: A magnetic stirrer and 50 milliliters of glacial acetic acid are placed in an iodine flask. The sample (usually about 0.2-0.5 g) for which the active bromine is to be determined is weighed and added to the flask containing the acetic acid. Water (50 milliliters) and aqueous potassium iodide (15% (wt/wt); 25 milliliters) are then added to the flask. The flask is stoppered using a water seal. The solution is then stirred for fifteen minutes, after which the flask is unstoppered and the stopper and seal area are rinsed into the flask with water. An automatic buret (Metrohm Limited) is filled with 0.1 normal sodium thiosulfate. The solution in the iodine flask is titrated with the 0.1 normal sodium thiosulfate; when a faint yellow color is observed, one milliliter of a 1 wt % starch solution in water is added, changing the color of the solution in the flask from faint yellow to blue. Titration with sodium thio sulfate continues until the blue color disappears. The amount of active bromine is calculated using the weight of the sample and the volume of sodium thiosulfate solution titrated. Thus, the amount of active bromine in a composition of this invention, regardless of actual chemical form, can be determined by use of this method.

Another standard method for determining active bromine is commonly known as the DPD test procedure. This method is well suited for determining very small amounts of active bromine in aqueous systems. The standard DPD test for determination of low levels of active halogen is based on classical test procedures devised by Palin in 1974. See A. T. Palin, “Analytical Control of Water Disinfection With Special Reference to Differential DPD Methods For Chlorine, Chlorine Dioxide, Bromine, Iodine and Ozone”, J. Inst. Water Eng., 1974, 28, 139. While there are various modernized versions of the Palin procedures, the recommended version of the test is fully described in Hach Water Analysis Handbook, 3rd edition, copyright 1997. The procedure for “total chlorine” (i.e., active chlorine) is identified in that publication as Method 8167 appearing on page 379, Briefly, the “total chlorine” test involves introducing to the dilute water sample containing active halogen, a powder comprising DPD indicator powder, (i.e., N,N′-diethyldiphenylenediamine), KI, and a buffer. The active halogen species present react(s) with KI to yield iodine species which turn the DPD indicator to red/pink. The intensity of the coloration depends upon the concentration of “total chlorine” species (i.e., active chlorine”) present in the sample. This intensity is measured by a colorimeter calibrated to transform the intensity reading into a “total chlorine” value in terms of mg/L Cl₂. If the active halogen present is active bromine, the result in terms of mg/L Cl₂ is multiplied by 2.25 to express the result in terms of mg/L Br₂ of active bromine.

In greater detail, the DPD test procedure is as follows:
1. To determine the amount of species present in the water which respond to the “total chlorine” test, the water sample should be analyzed within a few minutes of being taken, and preferably immediately upon being taken.
2. Hach Method 8167 for testing the amount of species present in the water sample which respond to the “total chlorine” test involves use of the Hach Model DR 2010 colorimeter. The stored program number for chlorine determinations is recalled by keying in “80” on the keyboard, followed by setting the absorbance wavelength to 530 nm by rotating the dial on the side of the instrument. Two identical sample cells are filled to the 10 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent. On the keypad, the SHIFT TIMER keys are depressed to commence a three minute reaction time. After three minutes the instrument beeps to signal the reaction is complete. Using the 10 mL cell riser, the blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂ is shown on the display within a few seconds. This is the “total chlorine” level of the water sample under investigation. By multiplying this value by 2.25, the level of active bromine in the water sample is provided.

The following examples are presented for purposes of illustration, and are not intended to impose limitations on the scope of this invention.

### EXAMPLE 1

Solutions of a sulfamate stabilized bromine chloride (SWG™ biocide; Albemarle Corporation), some diluted to achieve the desired concentrations, having different total bromine concentrations were siphoned by a pump at a 1:128 ratio to water to form the solution used to flush the drinking line system. The tests were performed on a poultry grow out farm in Texas. The samples were serially diluted and then plated to do the colony counts. Data on the amount of microbes present was collected before and after biocide treatment, as aerobic plate counts (APC) in colony-forming units per mL, in log₁₀. Results are summarized in Table 1. In Table 1, each value is an average of two data points, and total bromine concentrations are approximate.

TABLE 1

| | | | After | | After |
|---|---|---|---|---|---|
| Total | Contact | Initial | treatment | Initial | treatment |
| bromine | time | East line | East line | West line | West line |
| 1600 ppm | 4 hours | 4.55 | 2 | 3.03 | 1 |
| 400 ppm | overnight | 4.37 | 1.58 | 4.14 | 1.39 |
| 800 ppm | overnight | 4.71 | 2.0 | 4.70 | 3.39 |
| 1600 ppm | overnight | 4.72 | 1.5 | 5.2 | 0.65 |

Compatibility studies were performed on parts of the drinking line system and on the diaphragms of pumps typically used in a poultry house to ensure that at the concentrations used, the SWG™ biocide was compatible with these materials. The testing raised no concerns. Also, no issues regarding degradation of the drinking line system or the diaphragms of the pumps arose during the field test for which the results were reported above in Table 1.

### EXAMPLE 2

Experiments as in Example 1 were repeated at three different farms in northern Louisiana. Data on the amount of microbes present was collected before and after biocide treatment, as colonies per 100 mL. No dilutions of the samples were performed before filtering the water and plating it to do the colony counts. Results are summarized in Table 2. In Table 2, the reported concentrations of total bromine are approximate.

TABLE 2

| | Total | | | | |
|---|---|---|---|---|---|
| Farm | bromine | Contact time | Line | Initial¹ | After treatment¹ |
| A | 400 ppm | 3 hours | a | TNTC² | 57 |
| | | | b | 98 | 125 |
| | | | c | 43 | 45 |
| | | | d | TNTC | 21 |
| B | 800 ppm | 2.5 hours | e | TNTC | 110 |
| | | | f | TNTC | 42 |
| | | | g | TNTC | 69 |
| | | | h | TNTC | 61 |
| C | 1600 ppm | 6.5 hours | i | TNTC | 148 |
| | | | j | TNTC | 27 |
| | | | k | TNTC | 54 |
| | | | l | TNTC | 200 |

¹All initial values and after treatment values are reported as colonies per 100 mL.
²TNTC means too numerous to count, and signifies at least 300 or 400 colonies per 100 mL.

### EXAMPLE 3

Experiments were conducted at three different commercial hen farms in southern Mississippi. All three farms obtained their water for the drinking lines from private wells. Apparatus for collecting biofilms were installed in the chicken house drinking line systems at each farm. The apparatus contained metal and plastic washers on which biofilm could develop and which could be readily removed for analysis. The metal washers were made of 302 stainless steel, which is commonly used in nipple drinkers. The plastic washers were made of a vinyl material that is very similar to the material from which water transfer lines are made for commercial poultry grow out operations.

Each hen house had four apparatus installed to serve as controls (no treatment) and four apparatus installed for antimicrobial treatment. Apparatus were installed when the farms that had only eight weeks remaining before the birds were to be removed. After birds were removed, the control apparatus were removed from the drinking lines, placed in plastic bags, and shipped on ice to the University of Georgia for analysis. The remaining apparatus were left in place, and a solution of a sulfamate stabilized bromine chloride (SWG™ biocide; Albemarle Corporation), was introduced into the drinking lines at a ratio of 1:128 with water to form the solution used to flush the drinking line system. The diluted biocide remained in the water lines for about 24 hours. After the 24 hour period, the lines were flushed with water for 15 minutes. The remaining apparatus were removed from the drinking lines, placed in plastic bags, and shipped on ice to the University of Georgia for analysis. Apparatus arrived the next day and were either analyzed that same day, or kept on ice and analyzed the following day.

For the analyses, the metal and plastic washers were aseptically removed from the apparatus. Each washer had a “top” and “bottom” side designated, the top being the side that faced the head of the screw. Each washer was placed into a sterile petri dish with the top side up and marked to designate two half sides (a right and left side). The left side (top and bottom) was reserved for microscopic analysis, and the right side (top and bottom) was swabbed for colony forming unit (CFU) analysis. Samples were swabbed using sterile cotton swabs moistened with DE Neutralizing Buffer to neutralize the residues of the biocide. One mL of DE Neutralizing Buffer swab solution was diluted with 0.1% Peptone water (a minimal growth nutrient solution), and aliquots were plated onto duplicate plates of R2A Agar (low nutrient content) media using a spiral plater. One set of plates was incubated under aerobic (atmospheric) conditions at 25° C. for 7 to 10 days. The other set of plates were incubated under anaerobic (Gas-pack) conditions at 25° C. for 7 to 10 days. This culture medium and the incubation times used conform to that described in Standard Methods for the Examination of Water (heterotrophic plate count). Colonies that developed on the plates were counted, and the CFU/cm² (based on the area of the washer that was swabbed) was calculated.

Biofilm development on the untreated surfaces varied substantially, from as low as 286 CFU/cm² for one sample from one farm to a high of 2,500,000 CFU/cm² in a sample from another farm. For the treated systems, six had aerobic counts below the detection limits (4 CFU/cm² for plastic surfaces, 1 CFU/cm² for metal surfaces). Results are summarized in Table 3. In Table 3, the average number of CFU/cm² for is reported for both treated and control (untreated) samples. Some numbers are shown in Table 3 as “less than” because the average values include at least one sample that was below the detection limit. The results of these tests indicate that biocidal treatment was effective at inactivating the microbes in biofilms from the drinking line systems in each hen house.

TABLE 3

| | | | Untreated, in | | | |
|---|---|---|---|---|---|---|
| | | No. of | CFU/cm² | | Treated, in CFU/cm² | |
| Farm | Material | samples | Anaerobic | Aerobic | Anaerobic^{b} | Aerobic^{b} |
| D | Metal | 2; 4^{a} | 4300 | 97000 | <3 | <32 |
| | Plastic | 2; 4^{a} | 4600 | 7600 | <8 | <5 |
| E-1 | Metal | 2 | 190 | 1100 | <5 | <8 |
| | Plastic | 2 | 1700 | 8200 | 10 | 12 |
| E-2 | Metal | 2 | 670 | 18000 | <4 | 6 |
| | Plastic | 2 | 2000 | 55000 | 43 | <55 |
| F-1 | Metal | 2 | 9100 | 22000 | <25 | <317 |
| | Plastic | 2 | 10000 | 72000 | <5 | <263 |
| F-2 | Metal | 4 | 100 | 185000 | 7 | 9 |
| | Plastic | 4 | 480 | 828000 | 14 | 100 |

^{a}Two untreated samples and four treated samples.
^{b}The symbol < indicates that at least one sample was below the detection limit.

The log reduction data for two of the farms (D and E) is summarized in Table 4 below. Most of the log reductions are actually greater than indicated in Table 4 because the calculation includes samples below the detection limits. In addition, when the original level of biofilm is low, a high log reduction cannot be achieved due to the limits of detection. The log reduction data in Table 4 indicate no difference in effectiveness of the biocidal treatment between plastic and metal surfaces. Also, the log reductions are similar between poultry houses. It is noted that a 3 log reduction is equivalent to a 99.9% decrease in numbers.

TABLE 4

| | |
|---|---|
| Anaerobic | Aerobic |
| log reduction | log reduction |

| Farm | Metal | Plastic | Metal | Plastic |
|---|---|---|---|---|
| D | 3.2 | 2.8 | 3.5 | 3.2 |
| E | 2.1 | 1.8 | 3.2 | 3.0 |

Besides naturally-occurring impurities that may be present in water in general, such as well water or municipal water, the adjective “aqueous” also permits the presence in the water of dissolved salts that are formed in the course of forming a bromine-based microbiocide in the water, e.g., by reaction between bromine chloride and sodium sulfamate in an overbased aqueous solution. Also, “aqueous” permits the presence in the water of the amount of the halogen-based microbiocide itself to the extent that it may dissolve in the water, plus any dissolved reactant(s) that may remain after the reaction. In addition, the water may contain a few atoms that may dissolve from the vessel in which the reaction takes place, as well as air-borne impurities that may find their way into the water. The point here is that the term “aqueous” does not restrict the medium or solvent to absolutely pure water—the aqueous solution or medium or the like can contain what would normally be present and/or reasonably be expected to be present in it under the particular circumstances involved when employing ordinary common sense. Nor does the term “water” denote that it must be absolutely pure.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A method of controlling microbes in a drinking line system during which method poultry and/or swine are prevented from accessing the drinking line system via absence, which method comprises
I) contacting the drinking line system and an aqueous microbiocidal solution; and
II) flushing said drinking line system with water and/or an aqueous solution comprising one or more scale removers at least once after said contacting with said aqueous microbiocidal solution,
characterized in that said aqueous microbiocidal solution is formed from components comprising water and a concentrated aqueous biocidal solution selected from
A) an aqueous biocide solution which has a an active bromine content of bromine-containing species capable of biocidal activity of about 50,000 ppm or more, which solution is formed from components comprising water and
(i) bromine chloride or bromine chloride and bromine, with or without conjoint use of chlorine, and
(ii) overbased alkali metal salt of sulfamic acid and/or sulfamic acid, alkali metal base, and water,
wherein the relative proportions of (i) and (ii) are such that the atom ratio of nitrogen to active bromine is greater than 0.93, and wherein the pH of the composition is greater than 7, or
B) an aqueous biocide solution formed from components comprising water and
(i) at least one bromide source selected from ammonium bromide, hydrogen bromide, at least one alkali metal bromide, at least one alkaline earth metal bromide, and mixtures of any two or more of the foregoing,
(ii) a chlorine source,
(iii) optionally at least one inorganic base, and
(iv) optionally sulfamic acid and/or a metal salt of sulfamic acid, or
C) a combination of A) and B).

2. A method as in claim 1 further comprising flushing said drinking line system with water and/or an aqueous solution comprising one or more scale removers before said contacting with said aqueous microbiocidal solution.

3. A method as in claim 1 wherein said aqueous microbiocidal solution has a bromine residual in the range of about 50 to about 3200 ppm (wt/wt) as total bromine.

4. A method as in claim 1 wherein said aqueous microbiocidal solution has a bromine residual in the range of about 100 to about 2000 ppm (wt/wt) as total bromine.

5. A method as in claim 1 wherein the aqueous biocide solution is A).

6. A method as in claim 5 wherein said metal base of (ii) is sodium hydroxide.

7. A method as in claim 5 wherein said active bromine content of bromine-containing species is about 100,000 ppm or more.

8. A method as in claim 5 wherein said active bromine content of bromine-containing species is in the range of about 105,000 ppm to about 215,000 ppm.

9. A method as in claim 5 wherein said pH value is about 10 or greater.

10. A method as in claim 5 wherein said aqueous microbiocidal solution has a bromine residual in the range of about 50 to about 3200 ppm (wt/wt) as total bromine, and wherein said aqueous biocide solution has a an active bromine content of bromine-containing species of about 100,000 ppm or more and a pH value of about 13 or greater.

11. A method as in claim 1 wherein the aqueous biocide solution is B).

12. A method as in claim 11 wherein the aqueous biocide solution is formed from
a) water and (i) at least one bromide source selected from ammonium bromide, hydrogen bromide, at least one alkali metal bromide, at least one alkaline earth metal bromide, and mixtures of any two or more of the foregoing, (ii) at least one alkali metal hypochlorite and/or at least one alkaline earth metal hypochlorite, and (iii) an inorganic base, such that the pH of the aqueous biocide solution is greater than 7, or
b) water and (i) at least one bromide source selected from ammonium bromide, hydrogen bromide, at least one alkali metal bromide, at least one alkaline earth metal bromide, and mixtures of any two or more of the foregoing, and (ii) a solid chlorinating agent, and (iii) an inorganic base, such that the pH of the aqueous biocide solution is greater than 7, or
c) water and (i) at least one bromide source selected from ammonium bromide, hydrogen bromide, at least one alkali metal bromide, and at least one alkaline earth metal bromide, and mixtures of any two or more of the foregoing, (ii) a chlorine source, optionally (iii) at least one inorganic base, and (iv) sulfamic acid and/or a metal salt of sulfamic acid, or
d) a combination of any one or more of a) through c).

13. A method as in claim 12 wherein the aqueous biocide solution is a).

14. A method as in claim 12 wherein the aqueous biocide solution is b).

15. A method as in claim 12 wherein the aqueous biocide solution is c).

16. A method as in claim 15 wherein sulfamic acid and/or a metal salt of sulfamic acid is included.

17. A method as in claim 11 wherein (ii) is at least one alkali metal hypochlorite.

18. A method as in claim 17 wherein (i) is sodium hypochlorite.

19. A method as in claim 14 wherein (ii) is trichloroisocyanurate or sodium dichloroisocyanurate.

20. A method as in claim 13 wherein (iii) is sodium hydroxide.

21. A method as in of claim 13 wherein said pH value is about 10 or greater.

22. A method as in claim 15 wherein (iv) is sulfamic acid.

23. A method as in claim 11 wherein (i) is sodium bromide.

24. A method as in claim 1 wherein at least one dye and/or foaming agent is present in the aqueous biocide solution.
